(19) United States
(12) Patent Application Publication
(10) Pub. No.: US 2018/0318510 A1
(43) Pub. Date: Nov. 8, 2018

(54) INJECTION DEVICE

(71) Applicant: ARROW-MED DESIGN PTE. LTD., Singapore (SG)

(72) Inventor: Wah Leong Lum, Singapore (SG)

(21) Appl. No.: 15/773,777

(22) PCT Filed: Nov. 4, 2016
(86) PCT No.: PCT/SG2016/000019

§ 371 Date: May 4, 2018

(30) Foreign Application Priority Data
Nov. 5, 2015 (SG) 10201509159T

(51) Int. Cl. A61M 5/24; A61M 5/32; A61M 5/20

(52) U.S. Cl. CPC A61M 5/2466; A61M 5/326; A61M 5/2033; A61M 2005/2407; A61M 2005/2433; A61M 2005/3267; A61M 2005/2474; A61M 2005/2073

(57) ABSTRACT

An injection device comprises a barrel having a proximal end for a needle to protrude therefrom; a needle assembly having a double-pointed hollow needle and a biasing means operable to bias the double-pointed hollow needle away from the proximal end of the barrel; a cartridge; and a mechanism operable to cause the cartridge to be punctured by the first end of the needle, and to cause the second end of the needle to protrude from the proximal end of the barrel and to be locked in the protruded position.

### FIELD OF THE INVENTION

The present invention relates to an injection device. In particular, the injection device is suited, but not limited for administering subcutaneous injection to an individual.

### BACKGROUND ART

Individuals or patients suffering from diabetes typically require periodic injection of a medicament including insulin into their body to regulate their body's blood sugar level. Such periodic injections are usually administered by the individual themselves through known devices such as insulin pens, or by qualified medical professional(s) in a clinic or hospital.

In a hospital context, multi-dose vials containing vaccines are commonly used. Such multi-dose vials are shared amongst multiple patients. Typically, before administration of a dosage of insulin, a device such as a syringe with needle attached is used to extract the dosage from a multi-dose vial. To avoid the problem of contamination, usually in the form of cross-contamination, safety procedures such as the use of a sterilized new syringe and a needle for each different patient have to be strictly followed. Typically, such safety procedures, involving syringes and needles utilized in hospitals include their immediate disposal after administration of the medicinal dosage to patients. Therefore, the cost of such syringes and needles (collectively known as an injection device) are usually relatively low. As a result, such injection devices do not possess enhanced safety features for minimizing needle injury to the medical professional, unlike a comprehensive insulin pen for personal and multiple usage.

However, despite such safety procedures, failures in the adherence to such procedures, through human error, means that the possibility of contamination may still occur. In addition to contamination due to procedural lapses, the medical professional(s) may also be at risk of contamination due to accidental needle prick or injury arising from used needles. In view of the above, there remains a need to provide a cost-effective and simple injection device that could reduce the possibility of contamination and at the same time enhance safety for medical professionals.

It is therefore an object of the invention to attempt to alleviate the aforementioned problems.

### SUMMARY OF THE INVENTION

With the foregoing in view, the invention, in its broadest aspect resides in an injection device including:
a barrel having a proximal end for a needle to protrude therefrom;
a needle assembly having a double-pointed hollow needle and biasing means operable to bias the double-pointed hollow needle away from the proximal end of the barrel;
a cartridge; and
a mechanism operable to cause the cartridge to be punctured by the first end of the needle, and to cause the second end of the needle to protrude from the proximal end of the barrel and be locked in the protruded position.

Preferably the biasing means is a spring, and in such form, it is a compression spring.

Preferably, the mechanism is adapted to ensure that the action of puncturing and the protruding occur simultaneously.

Preferably, the barrel is so shaped and dimensioned as to cooperatively engage at least one of the mechanism, needle assembly or cartridge.

In such form, the cooperative engagement between the barrel and at least one of the mechanism, needle assembly and cartridge is provided by the presence of:
a) respective male and female screw threads;
b) a respective shoulder and a respective peripheral aspect adapted to sit on the shoulder;
c) movable catches, and catch receiving apertures; or
d) an interference fit.

Preferably, the mechanism forms part of the cartridge.

Preferably, the mechanism includes a rubber plug having a first portion and a second portion such that once inserted into the barrel, the second portion is arranged as an interference fit to urge the cartridge against the first end of the double-pointed hollow needle once the first portion rests on the circumferential periphery of the second end of the barrel.

Preferably, the barrel includes a first part and a second part, wherein the first part is adapted to receive and retain the needle assembly and the second part is adapted to receive and retain the cartridge and/or mechanism.

Preferably, the mechanism further enables the needle to be unlocked from its protruding position and in the unlocked position, the needle automatically retracts completely back into the barrel.

Preferably, the mechanism is adapted to ensure that the action of puncturing and the protruding occur simultaneously.

Preferably, the cartridge is pre-filled with a medicament.

Preferably, it is the distal end of the barrel that is so shaped and dimensioned as to cooperatively engage at least one of the mechanism, needle assembly or cartridge.

Preferably, the mechanism includes:
a. a rubber plug having a first portion and a second portion such that the second portion is arranged to urge the cartridge against an inner wall of the second part of the barrel; and
b. the coupling means such that as the first and second parts are coupled together, the cartridge is punctured by the first end of the double-pointed hollow needle, and the second end of the double-pointed hollow needle is protruded from the barrel and held in the protruded position.

Preferably, the cartridge is formed of a composite material and in such form, the composite material includes an inner layer made of glass and outer layer made of plastics material.

Preferably, the mechanism includes a. a rubber plug having a first portion and a second portion such that the second portion is arranged to urge the cartridge against an inner wall of the second part of the barrel; and b. the coupling means such that as the first and second parts are coupled together, the cartridge is punctured by the first end of the double-pointed hollow needle, and the second end of the double-pointed hollow needle is protruded from the barrel and held in the protruded position.

Preferably, the mechanism includes a pair of flaps arranged proximate to the second end of the barrel to prevent the cartridge from being ejected from the barrel due to the biasing means.

Although the cartridge could be ruptured by having thin walled sections strategically placed at one or more of its ends, preferably, the cartridge includes a number of demountable resilient seals that a needle can penetrate.

### BRIEF DESCRIPTION OF THE DRAWINGS

In order that this invention may be more readily understood and put into practical effect, reference will now be made to the accompanying sheets of drawings, which illustrate preferred embodiments of the invention by way of example only, wherein:

FIG. 1a shows an isometric view of an injection device in accordance with an embodiment of the invention;

FIG. 1b shows an exploded view of the injection device in accordance with the embodiment illustrated in FIG. 1a;

FIG. 2 illustrates the assembly and use of the device of FIG. 1a;

FIG. 3a shows an isometric view of an injection device in accordance with another embodiment of the invention;

FIG. 3b shows an exploded view of the injection device in accordance with the embodiment illustrated in FIG. 3a;

FIG. 4 illustrates the assembly and use of the device of FIG. 3a;

FIG. 5a shows an isometric view of an injection device in accordance with yet another embodiment of the invention;

FIG. 5b shows an exploded view of the injection device in accordance with the embodiment illustrated in FIG. 5a;

FIG. 6 illustrates the assembly and use the device of FIG. 5a;

FIG. 7a shows an exploded transparent view of the injection device in accordance with a final embodiment of the invention split into general sections labelled A, B and C;

FIG. 7b shows an exploded solid view of the injection device as shown in FIG. 7a;

FIG. 8a shows an isometric view of section A of the injection device shown in FIGS. 7a and 7b when it has been pre-assembled;

FIG. 8b shows a side elevation of section A of the injection device shown in FIGS. 7a and 7b when it has been pre-assembled;

FIG. 8c shows a more detailed view of a portion of the side elevation of FIG. 8b;

FIG. 9a shows an isometric view of section B of the injection device shown in FIGS. 7a and 7b when it has been pre-assembled;

FIG. 9a a side elevation of section B of the injection device shown in FIGS. 7a and 7b when it has been pre-assembled;

FIG. 10 illustrates the first step of the sequence of application of the injection device shown in FIGS. 7a and 7b—pre-assembled Section B about to be inserted into pre-assembled Section A;

FIG. 11 illustrates two views of the second step of the sequence of application of the injection device shown in FIGS. 7a and 7b—pre-assembled Section B being pushed into contact with pre-assembled Section A;

FIG. 12 illustrates two views of the third step of the sequence of application of the injection device shown in FIGS. 7a and 7b—protrusion of the needle and the locking of pre-assembled Section B into pre-assembled Section A;

FIG. 13 illustrates the fourth step of the sequence of application of the injection device shown in FIGS. 7a and 7b—the insertion of Section C into the locked components of pre-assembled Sections B and A;

FIG. 14 illustrates three views of the fifth step of the sequence of application of the injection device shown in FIGS. 7a and 7b—the locking of Section C to the pre-assembled Section B;

FIG. 15 illustrates three views of the sixth step of the sequence of application of the fully assembled injection device shown in FIG. 14—the drawing of a drug;

FIG. 16 illustrates three views of the seventh step of the sequence of application of the injection device shown in FIGS. 7a and 7b—after injection, the continued pushing in of the Section C plunger to release Section B from its locked position with Section A; and

FIG. 17 illustrates three views of the eighth step of the sequence of application of the injection device shown in FIGS. 7a and 7b—the withdrawal of the Section C plunger and Section B from Section A.

### DESCRIPTION OF THE PREFERRED EMBODIMENTS

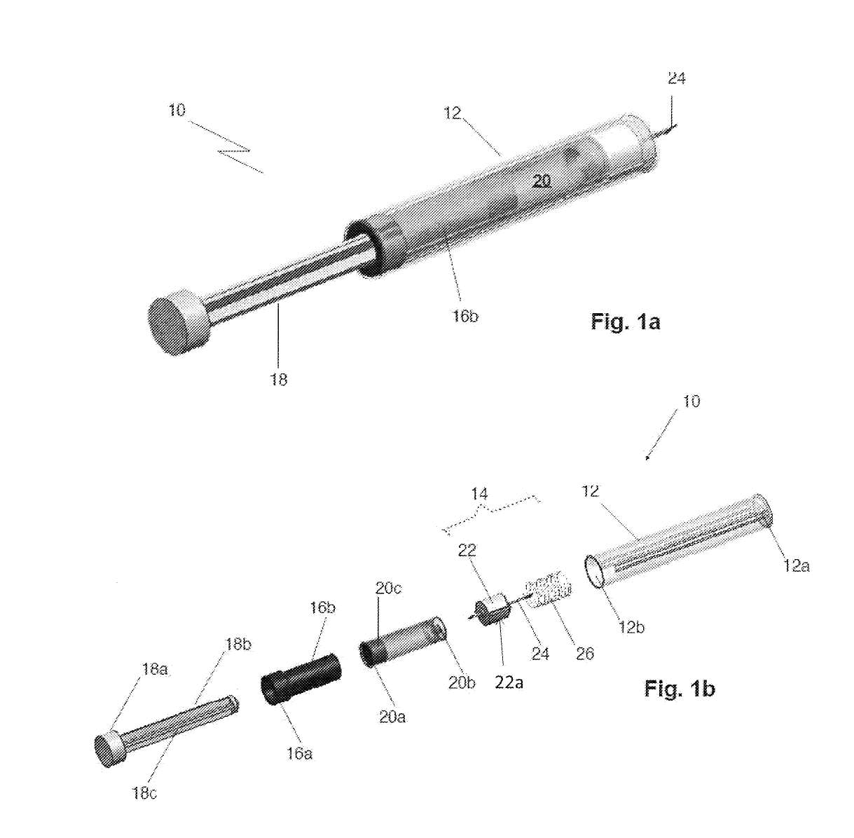

In accordance with an embodiment of the invention there includes an injection device 10. With reference to FIG. 1a and FIG. 1b, the device 10 includes a cylindrical barrel 12, a needle assembly 14, a plug 16 and a plunger 18. The device 10 further includes a cartridge 20, preferably containing a single dosage of a fluid composition for administration to an individual. Non-exhaustive examples of the fluid composition may be a vaccine or a medicament for treating diabetes having active ingredient insulin.

The cylindrical barrel 12 operates as a container to hold various components together.

Cylindrical barrel 12 is a one-part barrel 12 and includes a first end 12a and a second end 12b. First end 12a includes a hole for a needle 24 to protrude or retract therefrom.

Second end 12b includes an opening for receiving the needle assembly 14, the cartridge 20, the plug 16 and plunger 18. The use of the cylindrical barrel 12 greatly aids in the assembly of these various components as they can be slidably received within the barrel 12. Such simple assembly is readily suited to high volume automated production and represents a significant improvement in assembly costs compared to other devices.

The needle assembly 14 includes a needle holder 22, the needle 24, and a biasing means 26. The needle 24 is a double-pointed hollow needle suitable for insertion into the subcutaneous layer of an individual/patient. Some fluid compositions, such as the medicament for treating diabetes having active ingredient insulin, are usually injected subcutaneously i.e. in the sub-cutis lying between the cutis and a muscle membrane which cover the underlying muscles. If insulin is injected into the muscle tissue, it will be absorbed in the body too quickly and an unwanted drop in the blood sugar may result.

In order to prevent intramuscular injection of insulin, it is possible to inject insulin into a lifted skin fold. Alternatively, the needle 24 can be adapted to be suitable for insertion into the subcutaneous layer of an individual/patient by being short so that insulin may be injected directly without the skin fold. The needle 24 may be about 4 millimetres (mm) to about 16 mm, about 6 mm to about 16 mm, about 8 mm to about 12 mm, about 4 mm to about 10 mm, about 4 mm to about 8 mm or about 4 mm to about 6 mm.

The needle holder 22 is shaped to be similar to the cylindrical barrel 12 and has a diameter slightly smaller than the cylindrical barrel 12 for insertion into the cylindrical barrel 12. The needle holder 22 includes a slot 22a shaped and sized for the double-pointed needle 24 to insert therethrough and be held in position. In use, the hollow needle 24 allows fluid composition to flow from a punctured cartridge 20 to the needle 24.

The plug 16 includes a first portion 16a and a second portion 16b. The plug 16 is a rubber plug 16. The first portion 16a has a diameter similar to the diameter of the cylindrical barrel 12. The second portion 16b has a diameter slightly smaller than the diameter of the cylindrical barrel 12 such that when slidably received into the cylindrical barrel 12, the second portion 16b is inserted into the cylindrical barrel 12 and the first portion 16a sits snugly on the opening of the second end 12b. In operation, the plug 16 is operable to engage the cartridge 20 and activate the cartridge 20 for use.

Once the plug 16 is pushed in place the needle 24 is protruded from the first end 12a of the cylindrical barrel 12. The plunger 18 comprises a first portion 18a and a second portion 18b. The first portion 18a has a diameter similar to the diameter of the cylindrical barrel 12.

Extending from the first portion 18a is the second portion 18b that includes protrusions or flanges 18c operable to contact the second portion 16b of the plug 16 and facilitates sliding movement along the length of the cylindrical barrel 12. In operation, the plunger 18 is utilized to contact the cartridge 20 to urge the fluid composition out of the cartridge 20 into the needle 24. The cartridge 20 includes a first end 20a operable to interface with the second end 16b of the plug, and a second end 20b that can be punctured by the double-pointed needle 24 when a sufficient force is applied to the cartridge 20 against the needle 24. Cartridge 20 further includes a rubber seal 20c arranged at first end 20a. When the second end 20b of the cartridge is punctured, the plunger 18 contacts the rubber seal 20c and pushes the rubber seal 20c towards the second end 20b, thereby urging the fluid composition out of the cartridge 20.

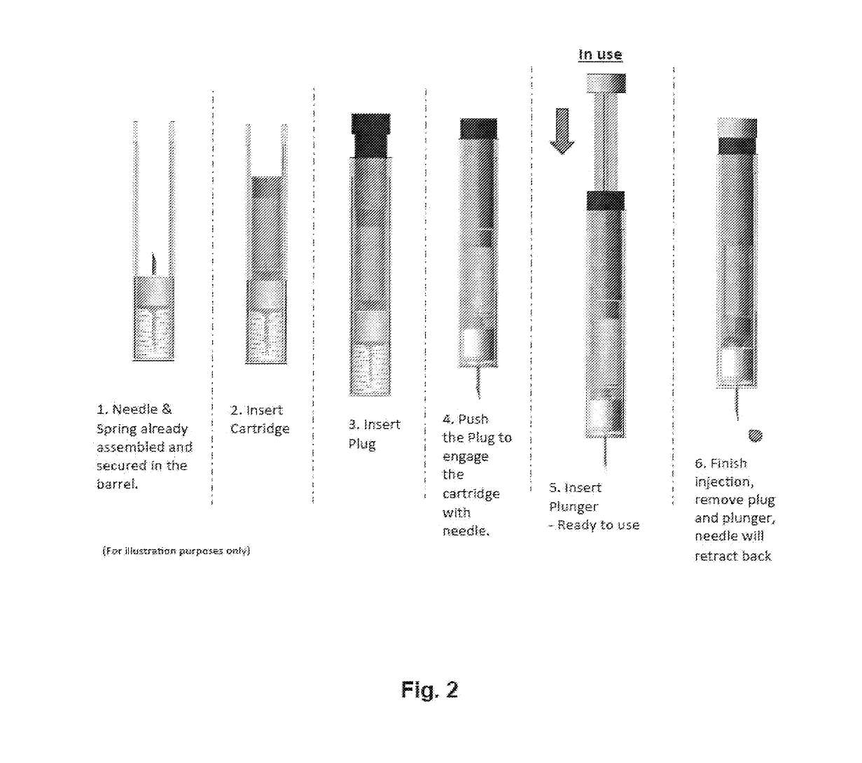

With reference to FIG. 2, the various components may be assembled to form the device 10 in a sequence as follows. The needle assembly 14 is first received within the cylindrical barrel 12 and arranged such that the needle 24 is prevented from protruding from the first end 12a of the cylindrical barrel 12 by the compression spring 26, which operates to urge the needle assembly 14 away from the first end 12a of the cylindrical barrel 12. As such, the risk of accidental needle prick or injury is eliminated. Furthermore, there is no need for an additional component, such as a safety shield, to prevent accidental needle prick or injury.

In this arrangement, one end of the spring 26 contacts the first end 12a of the cylindrical barrel 12, and the other end of the spring 26 contacts the needle holder 22. Next, the cartridge 20 containing the fluid composition is inserted into the cylindrical barrel 12. It is to be appreciated that the cartridge 20 contacts the needle assembly 14 but is not punctured in the absence of a large enough force to urge the cartridge 20 against the needle 24.

The plug 16 is next inserted into the cylindrical barrel 12. As the plug 16 is inserted into the cylindrical barrel 12:
i. The second portion 16b of the plug 16 urges against the first end 20a of the cartridge; and pushes the cartridge 20 towards the first end 12a of the cylindrical barrel 12;
ii. The cartridge 20 in turn pushes against the needle assembly 14 towards the first end 12a of the cylindrical barrel 12; and
iii. The spring 26 is compressed.

The above continues until one end of the needle 24 is protruded from the first end 12a, and the other end of the needle 24 punctures the cartridge 20, thereby allowing fluid to flow from the cartridge 20 to the hollow needle 24. As the plug 16 is pushed within the cylindrical barrel 12, the interference fit of the second portion 16b of the plug 16 as it contacts the inner surface of the cylindrical barrel 12 prevents the spring 26 from pushing the needle assembly 14 out of position or causing the protruded needle 24 to be retracted.

Once one end of the needle 24 is protruded from the cylindrical barrel 12 and the other end of the needle punctures the cartridge 20, the device 10 is ready for usage, i.e. for injecting the fluid composition such as insulin into an individual. In use, the plunger 18 is pushed into the cylindrical barrel 12 through the plug 16 and contacts the rubber brush/seal 20c. As the seal 20c moves, fluid composition is urged out of the cartridge 20 to the needle 24. Upon usage, the plug 16 and plunger 18 may be simply removed from the cylindrical barrel 12.

Once the plug 16 is removed, the spring 26 operates to urge the needle assembly 14 away from the first end 12a of the cylindrical barrel 12. The needle 24 then simultaneously retracts from the first end 12a as the needle assembly 14 is urged away from the first end 12a. The device 10 may then be disposed/handled safely with-out the needle protruding from the cylindrical barrel 12, hence minimizing the risk of needle pricks or injury.

Further, the risk of contamination is also reduced because of the avoidance of multi-dosage vials. Instead, a cartridge 20 including a single dosage suitable for a patient is used. The components used are also relatively cheap, and could be easily mass-produced. For instance, the plunger 18, plug 16 and cylindrical barrel 12 may be made from synthetic plastics material, and thus can be disposed of economically after a single use. Each device 10 can also be disposable.

Consequently, the device 10 is commercially viable to make, such that the production costs are reasonable compared with the costs of other injection devices. This contrasts with other devices, the cost of which must be supported by repeated reuse. In addition, a person skilled in the art would appreciate that each reuse requires sterilization, which adds further to the cost of other devices.

Furthermore, before being sterilised, the spent cartridge and notably, the used needle have to be removed and disposed of, which exposes the user to the risk of needle pricks or injury. In addition, the vectors of some very serious diseases are known to be resistant to regular sterilisation. In contrast, device 10 is inexpensive, safe and convenient to use. In accordance with another embodiment of the invention there includes an injection device 100. Instead of having a one-part barrel 12 for holding the needle assembly, plug and plunger, the cylindrical barrel 12 is provided with coupling means 130 for coupling with a needle cap assembly 140.

In the device 100, the additional needle cap assembly 140 is introduced to house the needle assembly 14. This embodiment is suitable for use with a prefilled insulin pen.

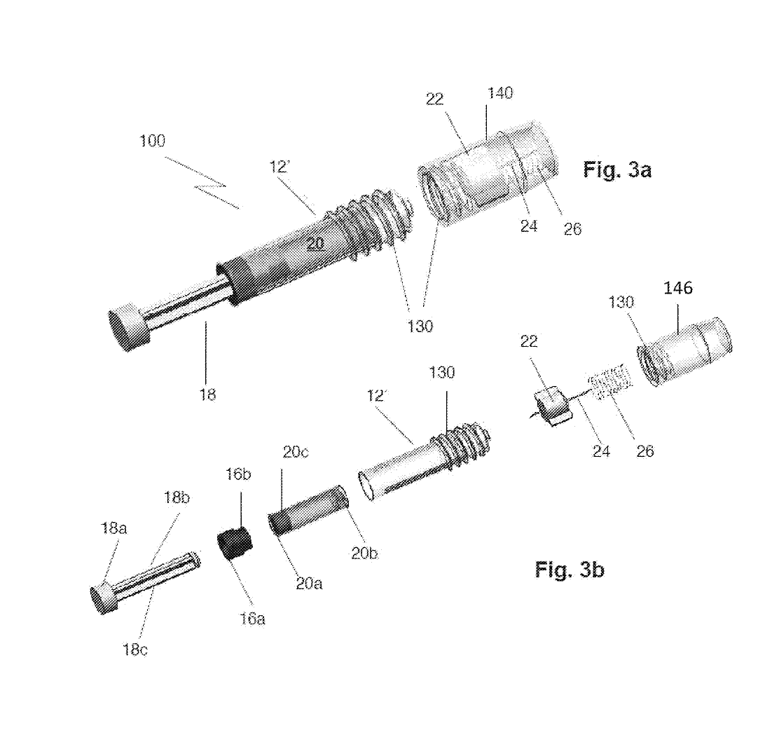

With reference to FIG. 3a and FIG. 3b, wherein like numerals reference like parts, the cylindrical barrel 12 is used to receive a cartridge 20 containing a single dosage of a fluid composition. The fluid composition may be a vaccine or a medicament for treating diabetes having the active ingredient insulin. The needle cap 142 includes a hole for one end of the double-pointed hollow needle 24 to protrude therefrom.

The other end of the needle 24 is aligned with a hole formed on the modified barrel 12′ such that when the needle cap assembly 140 is coupled to the cylindrical barrel 12′, the other end of the needle 24 will puncture the cartridge 20 during the coupling process.

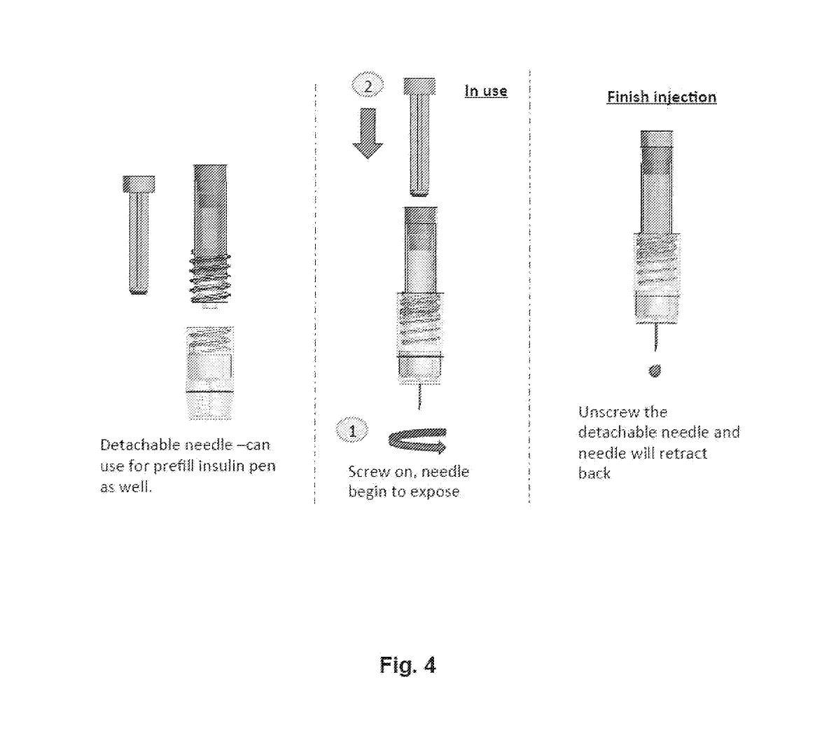

The modified barrel 12′ and the needle cap 142 each include cooperating coupling means 130 in the form of male and female screw threads. With reference to FIG. 4, the device 100 is assembled in a sequence as follows. The cartridge 20 containing the fluid composition is inserted into the modified barrel 12′ and rests against an end proximate to the screw thread 130 of the modified barrel 12′. The plug 16 provided with an interference fit is next inserted into the modified barrel 12′ to hold the cartridge 20 in place.

The needle cap assembly 140 houses the needle holder, needle and the compression spring arranged such that as the needle cap 142 is coupled with the modified barrel 12′ via the screw threads 130, one end of the needle protrudes from the needle cap 142, while the other end of the needle punctures the cartridge 20 and compresses the spring 26.

The device 100 is ready for usage. In use, the plunger 18 is pushed through the plug 16 and contacts the rubber brush/seal 20c to urge the fluid contents out of the cartridge 20 to the needle 24. Once used, the needle cap assembly 140 may be decoupled from the cylindrical barrel 120.

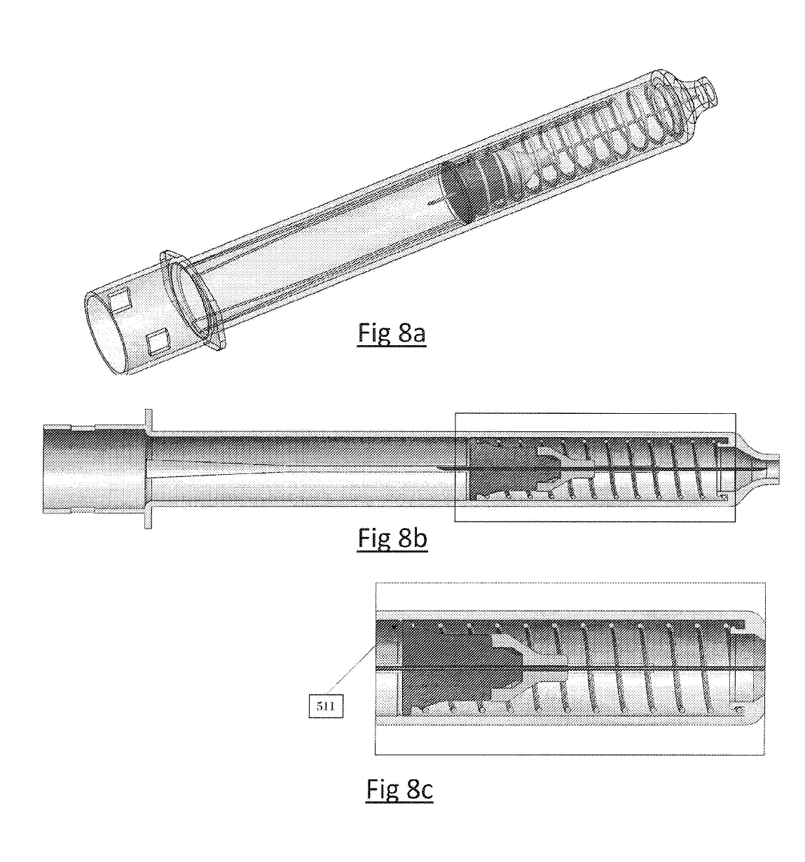

Upon decoupling, the needle assembly 14 inclusive of the entirety of the needle 24 is retained wholly within the needle cap 146 through the advantageous location of the same ‘shoulder-type’ arrangement within the needle cap 146, as shown in FIG. 8c. The device 100 may then be handled or disposed safety without causing accidental needle pricks or injuries.

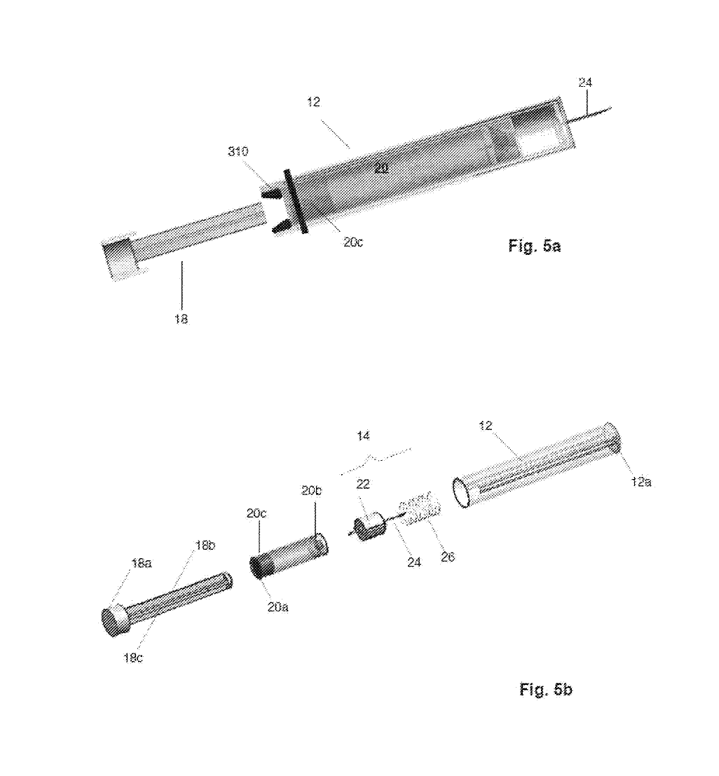

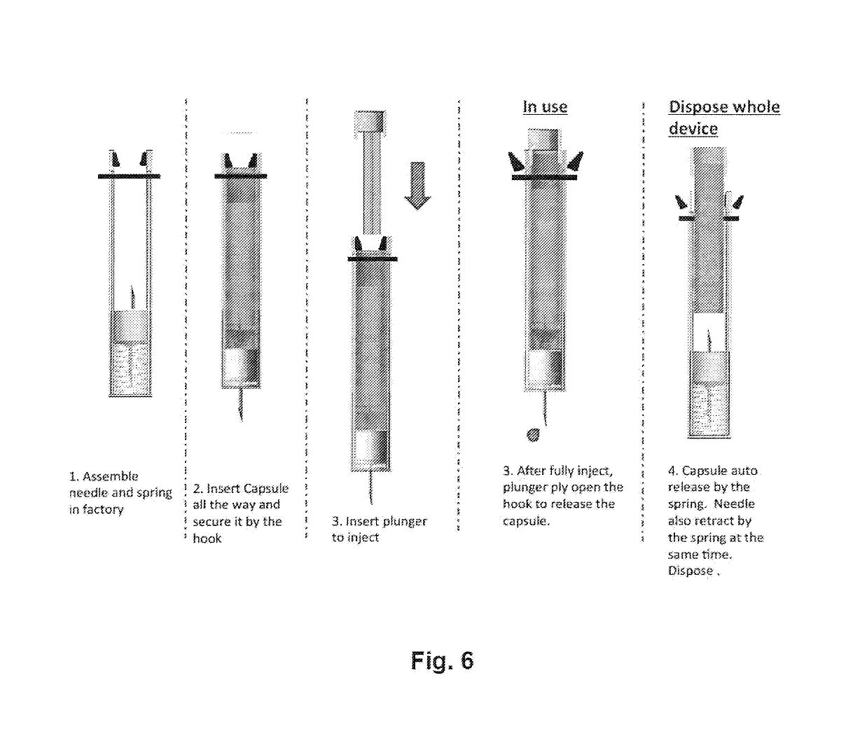

Comparing the device 100 and device 10, if the overall length of the device is to be the same, with a shorter modified barrel 12′, the second portion 16b of plug 16 to hold the cartridge 20 in place will have a shorter length. FIG. 5a and FIG. 5b illustrates yet another embodiment of the invention, which omit the requirement of a plug 16. In the embodiment depicted in FIG. 5a and FIG. 5b, the assembly is similar to the arrangement shown in FIG. 1a and FIG. 1b except that the open-end 12b of cylindrical barrel 12 includes a resilient means 310.

Resilient means 310 includes two flaps 310a, 310b positioned proximate to the open-end 12b. As the cartridge 20 is inserted into the cylindrical barrel 12, the outer surface of the cartridge 20 pushes against the flaps 310a, 310b, which in turn are compressed and pushed towards the inner walls of the cylindrical barrel 12. When the cartridge 20 is fully inserted, the cartridge 20 no longer exerts a lateral force on the flaps 310a, 310b which push the flaps 310a, 310b against the inner wall of the cylindrical barrel 12.

The flaps in turn are no longer compressed and therefore are able to return to its original shape, thereby resisting the top surface of the cartridge 20 from sliding out of the cylindrical barrel 12 due to the force acted by spring 26. The flaps 310a, 310b are dimensioned such that they allow the portion 18b of plunger 18 to easily enter the cylindrical barrel 12 to discharge the fluid composition, but once the plunger is fully inserted, the portion 18a of the plunger 18 act against the flaps 310a, 310b to ply open the same.

The cartridge 20 may then be easily ejected out from the cylindrical barrel 12 due to the action of spring 26. At the same time, the needle 24 is also automatically retracted due to the same action by spring 26.

In some embodiments, a hook or stopper mechanism may be formed on the cartridge 20, the hook or stopper mechanism operable to perform similar function as the flaps 310. Such a mechanism functions to cause the cartridge to be punctured by the first end of the double-pointed hollow needle and to simultaneously cause the second end of the double-pointed hollow needle to protrude from the cylindrical barrel 12 and be held in the protruded position. If the overall length of the device of embodiment FIG. 5b is of a similar length to the length of device 10, 100, then in the absence of a plug 16, it is to be appreciated that the length of the cartridge 20 may be longer to achieve a similar activation of the needle 24.

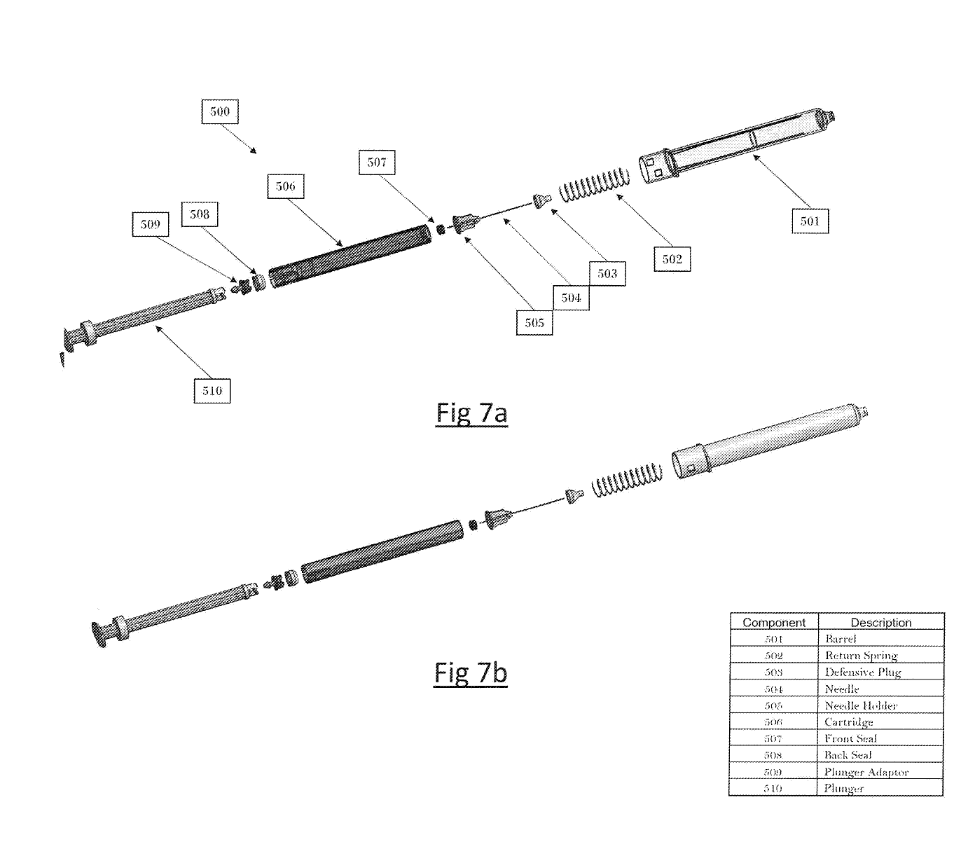

In some embodiments, (see FIG. 7a onwards) the cartridge 20 may not be filled with any fluid composition or vaccine. In these embodiments, there will be an additional step of filling the cartridge 20 with the necessary vaccine or fluid composition before inserting the filled cartridge 20 into the cylindrical barrel.

In some embodiments, the cartridge 20 may be formed of a composite material including an inner layer made of glass and outer layer made of plastics material. In operation, the inner layer of the cartridge 20 is in contact with the fluid composition. Such an arrangement is advantageous as certain medicament/vaccine may react with plastics material, and the inner glass layer effectively prevents any unintended chemical reaction to take place between the fluid composition and the outer plastic layer. The outer plastics material layer functions as a covering to protect the inner fragile glass layer.

FIG. 7a shows a further embodiment of the invention, an injection device generally referenced 500, which includes:
1) an elongate cylindrical barrel 501, a needle assembly including a compression spring 502, a defensive plug 503, a double ended hollow needle 504 and a needle holder 505 adapted to seal the needle 504 in place, (collectively, forming Section A of the device 500);
2) an elongate cylindrical empty cartridge formed from a cylindrical tube 506, which is sealed by a front resilient seal 507 at its proximal end, and a rear resilient seal 508 at its distal end, together with a plunger adaptor 509, (collectively these components form the sealed cartridge or Section B of the device 500); and
3) a plunger 510.

The barrel 501 formed from Plastics material is provided with an internal circular shoulder 511 to prevent the needle assembly components 502-505 from dropping out once inserted into the barrel 501 and initially pushed beyond the shoulder 511, as the periphery of the needle holder 505 now sits on the shoulder 511 at this point. FIGS. 8a-8c show the pre-assembly of the Section A components and illustrate the relationship between the shoulder 511 and the periphery of the needle holder 505.

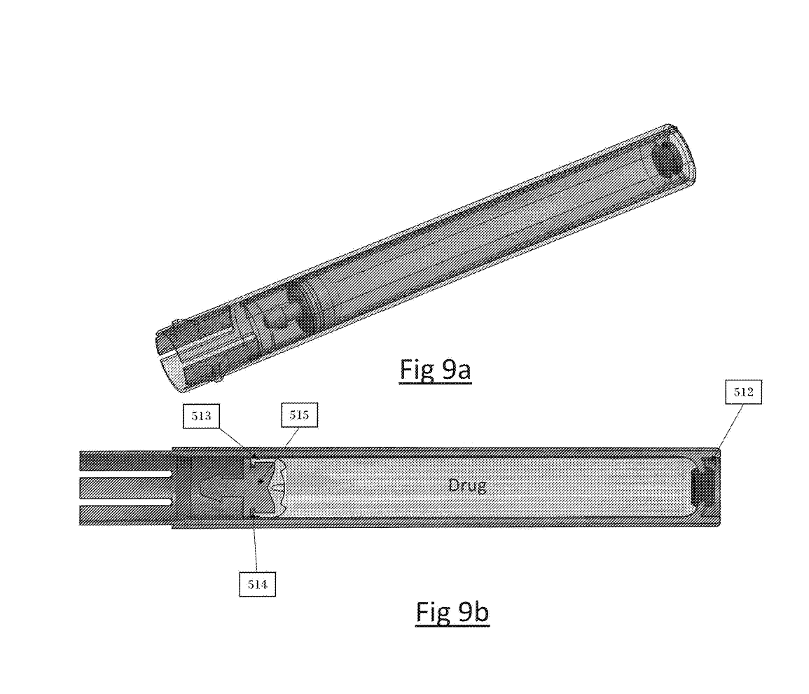

FIGS. 9a and 9b shows pre-assembled Section B, which includes the front and rear seals 507 and 508, plugged into the cylindrical tube 506 to seal and prevent a fluid drug it will later contain from escaping. The cylindrical tube 506 is provided with a curved front circular shoulder 512, adapted to receive the neck of the front seal 507 and a circular shoulder 513 adapted to receive a cooperating circular recess of the rear seal 508.

The rear seal 508 and the plunger adaptor 509 cooperate with each other, to complete the pre-assembly, the former providing a peripheral shoulder 514 to lock the plunger adaptor 509 in place and the latter providing the resilient body 515 to bias the circular recess against the shoulder 513 locking the rear seal 508 in place.

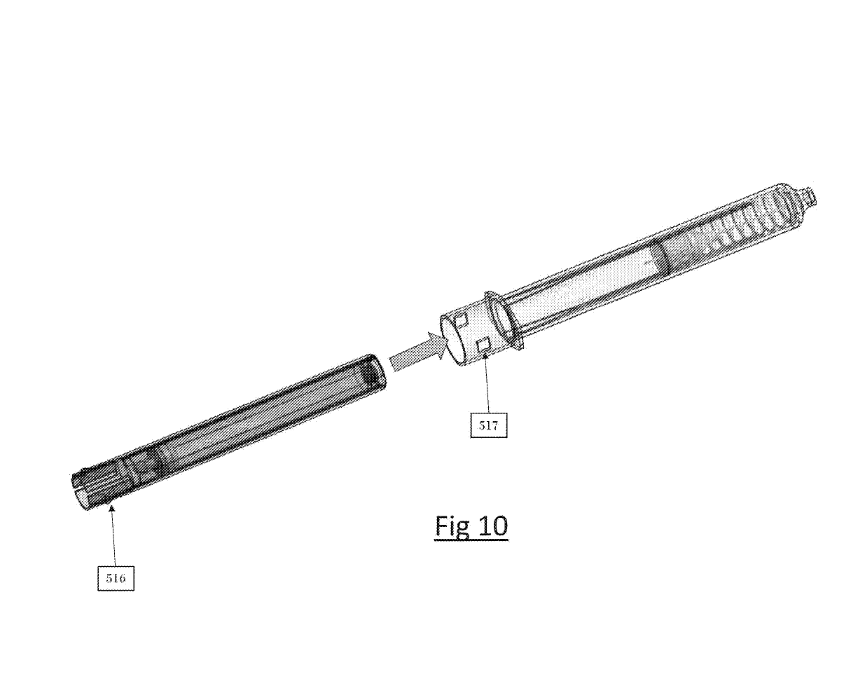

FIG. 10 shows the pre-assembled Section B about to be inserted into the pre-assembled Section A.

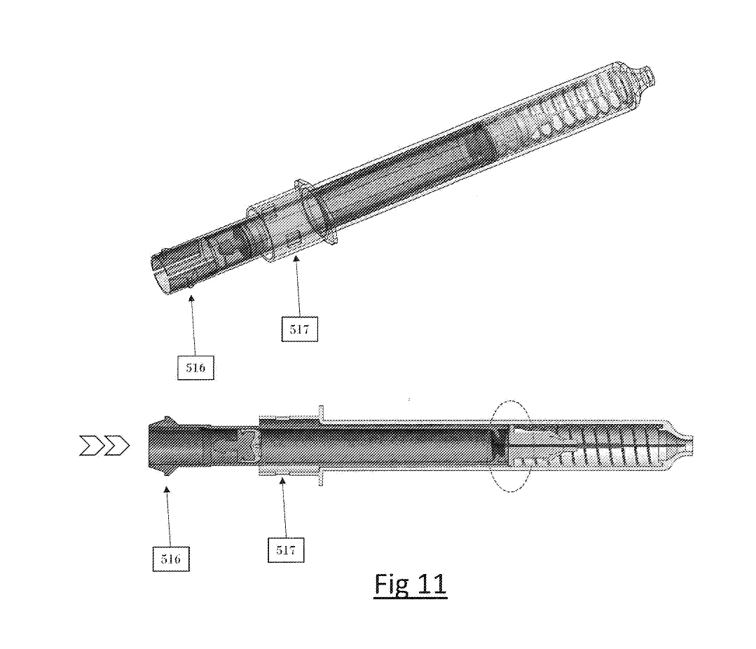

From FIG. 11, it is clear that as Section B slidably enters Section A, the end of the double ended needle 504 facing the front seal 507 will eventually puncture the seal 507, the compression spring 502 now beginning to act as a bias against further inward movement of Section B and the compression of the spring 502 also causing the other end of the needle 504 to commence exiting the proximal end of the barrel 501.

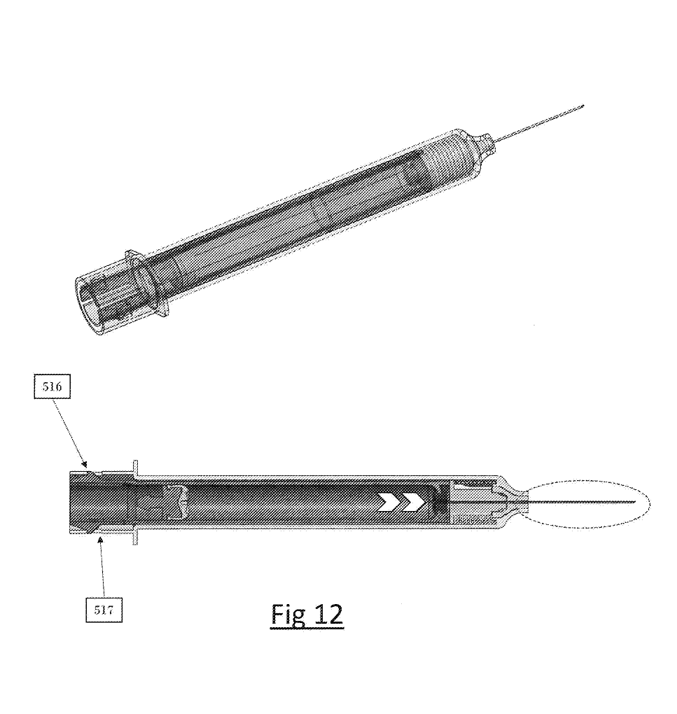

Resilient catches 516, and apertures 517 located at the distal ends of barrel 501 and tube 506 respectively, cooperate to cause Section B to slidably engage with section A (as Section B is further pushed into Section A) to lock Section B into Section A (see FIG. 12).

At this point, the compression of the compression spring 502 is at a maximum with the consequence that respective ends of the needle 504 are fully inserted through the seal 507 and fully extended out of the proximal end of the barrel 501. The resilience of the catches 516, is provided by the fact that distal end of the tube 506 containing the catches 506 is provided with a plurality of elongate channels 517 splitting the distal end of the tube 506 into sections which are able to flex and thus enable the catches 516 to flex radially.

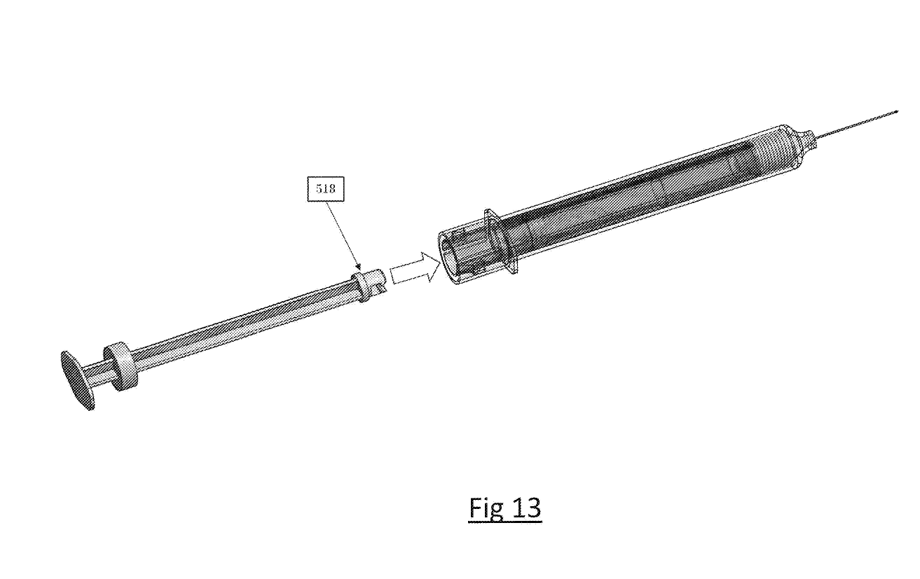

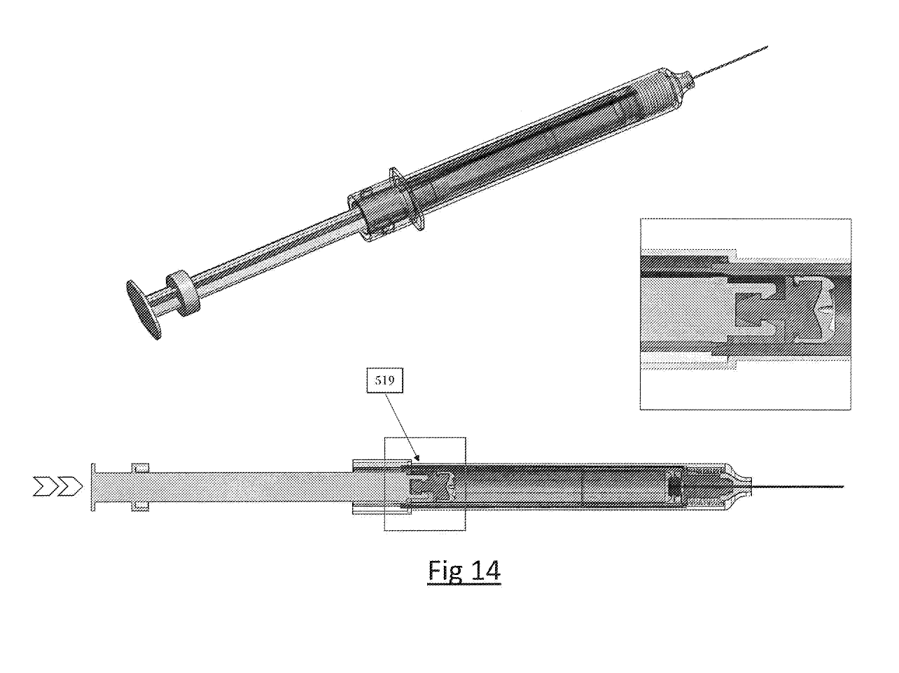

From FIGS. 13 and 14, the plunger 510 at its proximal end contains a pair of jaws 518 adapted to engage and lock with a corresponding cooperating portion 519 of the plunger adaptor 509 in a snap fit arrangement. Once this locking has been achieved, filling of the injection device 500 with a drug can commence.

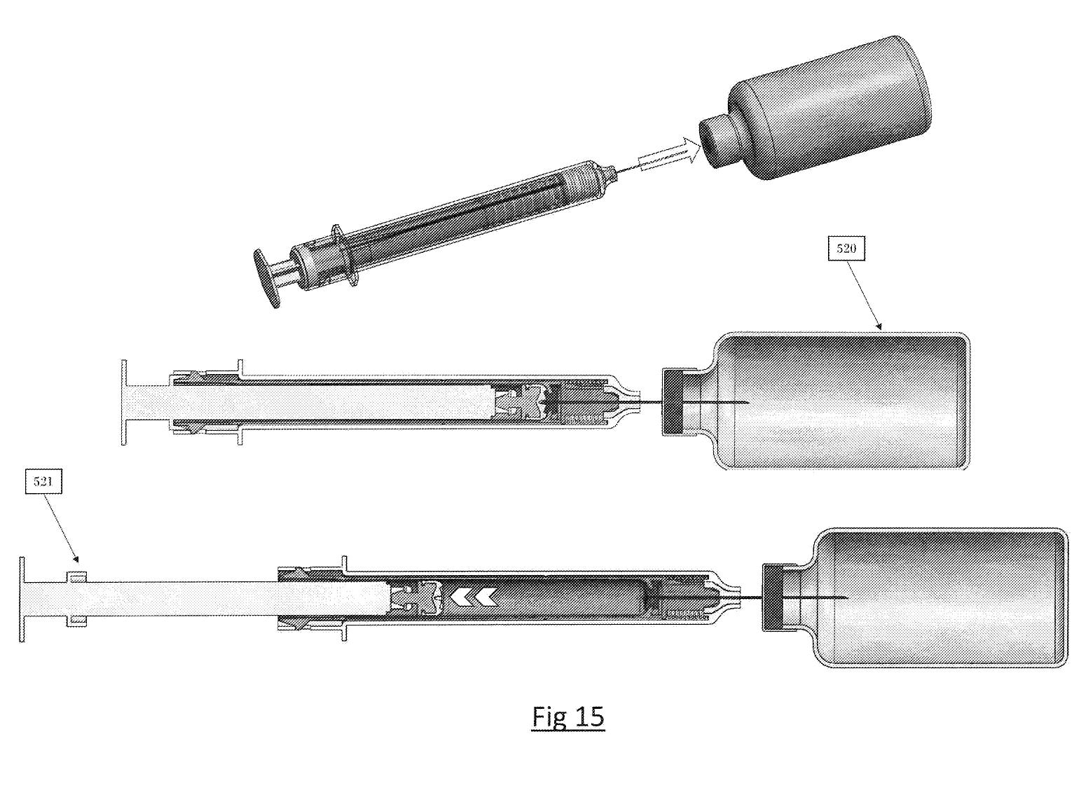

FIG. 15 shows a sequence of actions involving the fully assembled injection device consisting of Sections A, B and C, drawing a drug from a septum sealed container 520. It will be noted that towards the distal end of the plunger 510 is provided with a coaxial circular projection 521 parallel with the principal axis of the plunger 510.

During the drug filling of the injection device 500, Section C is pushed into Section B until the end of the projection 521 begins to contact the catches 516, at which point further pushing of Section C into Section B is stopped. The needle 504 projecting from the proximal end of the barrel 501 is then inserted into the container 520 through the septum. Section C is then pulled back relative to section Band fluid drug is drawn into the injection device 500. The needle 504 is then withdrawn from the container 520.

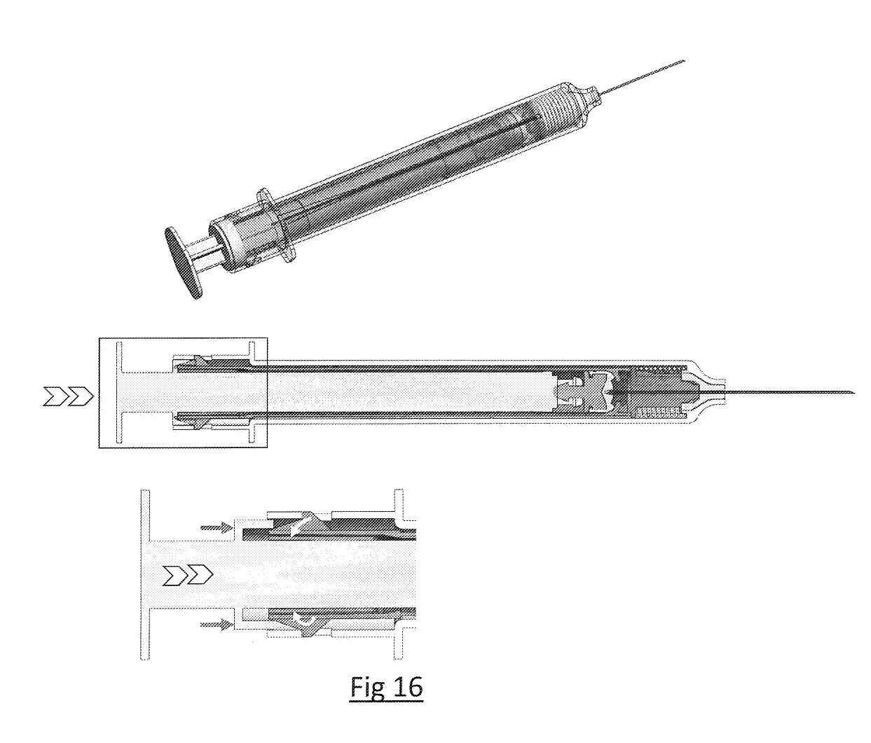

After injection of the drug, FIG. 16 depicts the next stage of the procedure which will reduce the risk of any form of contamination. Section C is now fully pushed into Section B and in the final part of this action, the ends of the projection 521 engage with and force the catches 516 to move radially inwards, unlocking Section B from Section A. Once unlocked, the spring 502 in its compressed state, is able to relax back into its non-compressed state and as it does so, it acts as a bias pushing Section B relative to and out of Section A. A partial vacuum retains Section C within Section B as it withdraws.

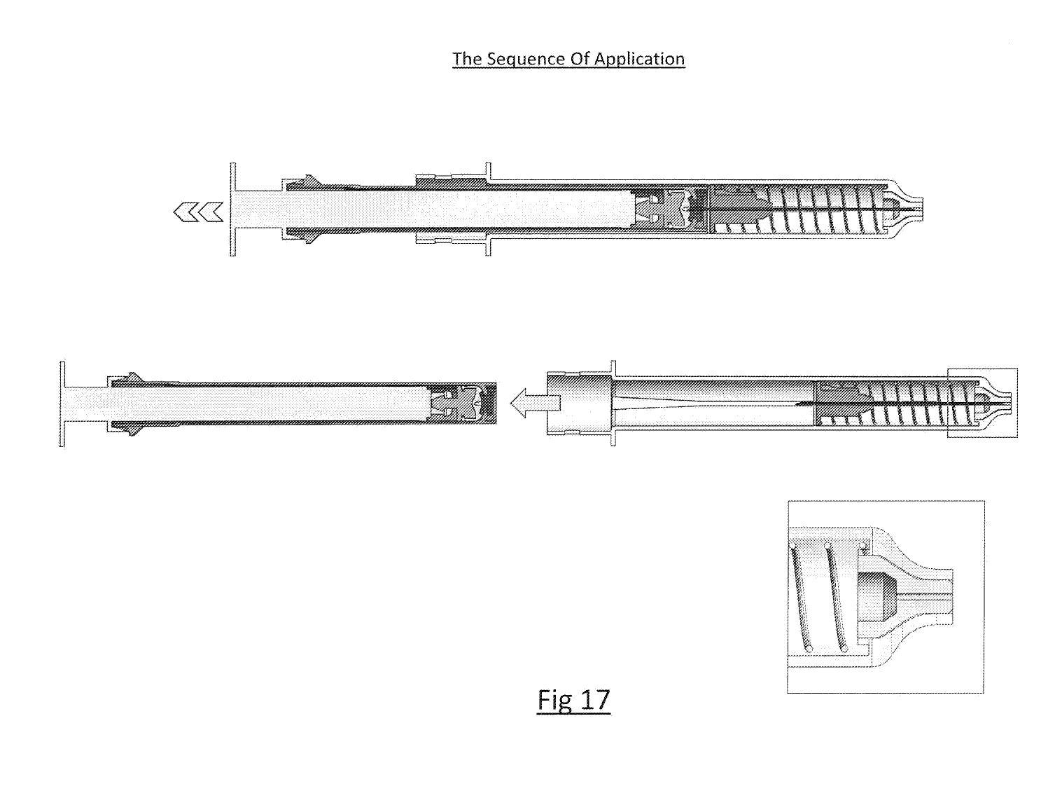

At the same time, the spring 502 also retracts the needle 504 from the proximal end of the barrel 501 back fully into the body of the barrel 501, as depicted in FIG. 17. It is to be appreciated that the various components of the embodiments described including for example plunger 18, plug 16, cartridge 20, needle assembly 14, barrels 12, 12′, needle cap assembly 140, resilient means 310 may be mixed and matched to form further embodiments.

The various components, singly or in plurality may also be provided in the form of a kit for users.

In the various embodiments described, it is to be appreciated that the plug 16, resilient means 310 and the coupling means 130 function singly or in combination as a mechanism operable to cause the cartridge to be punctured by the first end of the double-pointed hollow needle, and to simultaneously cause the second end of the double-pointed hollow needle to protrude from the barrel and be held in the protruded position. Such an arrangement is advantageous in that the needle in the injection device is generally at the retracted state until activated when the needle is protruded. Once utilized, the needle is retracted and the device may be safely disposed.

It is also to be understood that the above embodiments have been provided only by way of exemplification of this invention, and that further modifications and improvements thereto, as would be apparent to persons skilled in the relevant art, are deemed to fall within the broad scope and ambit of the present invention described herein.

It would be further appreciated that although the invention covers individual embodiments, it also includes combinations of the embodiments discussed. Therefore, features described in one embodiment not being mutually exclusive to a feature described in another embodiment may be combined to form yet further embodiments of the invention.

1. An injection device including:
a barrel having a proximal end for a needle to protrude therefrom;
a plunger;
a needle assembly having a double-pointed hollow needle and a biasing means operable to bias the double-pointed hollow needle away from the proximal end of the barrel; and
a cartridge;
their arrangement being concentric once assembled to form the device, with the biasing means being located at the proximal end of the barrel; and
a mechanism operable to cause the cartridge to be punctured by a first end of the needle, and to cause a second end of the needle to protrude from the proximal end of the barrel and to be locked in a protruded position prior to any movement of the plunger, wherein the mechanism is adapted to ensure that the action of puncturing and the protruding occur simultaneously.

2. The injection device as claimed in claim 1, wherein the mechanism further enables the needle to be unlocked from its protruding position and in an unlocked position, the needle automatically retracts completely back into the barrel.

3. The injection device according to claim 1, wherein the cartridge is pre-filled with a medicament and the needle assembly including the double-pointed hollow needle and biasing means adapted to initially be housed completely within the barrel.

4. The injection device according to claim 1, wherein the barrel is so shaped and dimensioned as to cooperatively engage at least one of the mechanism, needle assembly or cartridge.

5. The injection device according to claim 4, wherein the cooperative engagement between the barrel and at least one of the mechanism, needle assembly and cartridge is provided by a presence of:
e) respective male and female screw threads;
f) a respective shoulder and a respective peripheral aspect adapted to sit on the shoulder;
g) movable catches, and catch receiving apertures; or
h) an interference fit.

6. The injection device according to claim 4, wherein the mechanism forms part of the cartridge.

7. The injection device according to claim 4, wherein it is a distal end of the barrel that is so shaped and dimensioned as to cooperatively engage at least one of the mechanism, needle assembly or cartridge.

8. The injection device according to claim 5, wherein the mechanism includes a resilient plug to enable the interference fit with the barrel.

9. The injection device according to claim 1, wherein the mechanism includes:
a. a rubber plug having a first portion and a second portion such that the second portion is arranged to urge the cartridge against an inner wall of the barrel; and
b. a coupling means such that as the first and second portions are coupled together, the cartridge is punctured by the first end of the double-pointed hollow needle, and the second end of the double-pointed hollow needle is protruded from the barrel and held in the protruded position.

10. The injection device according to claim 1, wherein the mechanism includes a pair of flaps arranged proximate to a second end of the barrel to prevent the cartridge from being ejected from the barrel due to the biasing means.

11. The injection device according to claim 1, wherein the cartridge is formed of a composite material.

12. The injection device as claimed in claim 11, wherein the composite material includes an inner layer made of glass and outer layer made of plastics material.

13. The injection device according to claim 1, wherein the cartridge includes a number of demountable seals.

14. The injection device according to claim 2, wherein the cartridge is pre-filled with a medicament.

15. The injection device according to claim 14, wherein the barrel is so shaped and dimensioned as to cooperatively engage at least one of the mechanism, needle assembly or cartridge.

16. The injection device according to claim 15, wherein the cooperative engagement between the barrel and at least one of the mechanism, needle assembly and cartridge is provided by a presence of:
e) respective male and female screw threads;
f) a respective shoulder and a respective peripheral aspect adapted to sit on the shoulder;
g) movable catches, and catch receiving apertures; or
h) an interference fit.

17. The injection device according to claim 2, wherein the mechanism includes a pair of flaps arranged proximate to a second end of the barrel to prevent the cartridge from being ejected from the barrel due to the biasing means.

18. The injection device according to claim 17, wherein the cartridge is formed of a composite material.

19. The injection device according to claim 18, wherein the composite material includes an inner layer made of glass and outer layer made of plastics material.

20. The injection device according to claim 19, wherein the cartridge includes a number of demountable seals.
